(19) United States
(12) Patent Application Publication
(10) Pub. No.: US 2022/0117223 A1
(43) Pub. Date: Apr. 21, 2022

(54) ANTIBACTERIAL ANTIVIRAL TREATED FABRICS

(71) Applicant: MIND CREATIONS, LLC, MIAMI, FL (US)

(72) Inventor: MARIA CANDELARIA GUERREIRO, CHUBUT (AR)

(73) Assignee: MIND CREATIONS, LLC, MIAMI, FL (US)

(21) Appl. No.: 17/490,190

(22) Filed: Sep. 30, 2021

Related U.S. Application Data
(60) Provisional application No. 63/094,636, filed on Oct. 21, 2020.

(51) Int. Cl. A01N 25/34; A01N 59/00; A61L 2/18; D06M 23/00; D06M 16/00

(52) U.S. Cl. CPC A01N 25/34; A01N 59/00; A61L 2202/26; D06M 23/00; D06M 16/00; A61L 2/18

(57) ABSTRACT

The invention discloses a method to impart a substrate fabric with antiviral or antibacterial properties consisting in implanting copper and/or silver nanoparticles into natural and synthetic fabrics. A plurality of products manufactured utilizing the fabrics processed according to the disclosed methods for use where antiviral and antibacterial properties are required, such as hospital and other aseptic places.

### CROSS REFERENCE TO RELATED APPLICATIONS

This application claims priority to U.S. Provisional Application 63/094,636, titled ANTIBACTERIAL ANTIVIRAL TREATED FABRICS, filed on Oct. 21, 2020, the entire contents of which are herein incorporated by reference.

### FEDERALLY SPONSORED RESEARCH

Not Applicable

### SEQUENCE LISTING OR PROGRAM

Not Applicable

### FIELD OF INVENTION

The present invention is related to a method to impart antiviral and antibacterial characteristics to a fabric substrate and to manufacture products having those characteristics for use in aseptic places.

The present invention comprises a plurality of fabrics upon which a proprietary chemical treatment confers them antibacterial and antiviral properties. These treated fabrics can be manufactured and then incorporated into a plurality of products for use in circumstances whereby pathogens that may come in contact with humans can be killed beforehand.

### BACKGROUND OF THE INVENTION

Viruses and bacteria inhabit and share areas that usually are also inhabited by humans. Whether on furniture surfaces, floating in air, or as part of liquids or food ingested by humans, viruses and bacteria seem omnipresent. Many of these microscopic organisms play a positive, even essential, role in a human's life; however, often an imbalance in the relationship converts them into pathogens.

Humans developed bio-responses to defend themselves against viruses and learned to have a symbiotic existence with some bacteria. While the preceding generalization may apply to many humans, others with immunodeficiencies need to be isolated from pathogens that may harm or even kill the compromised human.

Various methods exist to control the contact between pathogens and humans. In addition to total isolation, pathogens may be killed by chemical, biological, electromechanical, luminescent means, and combinations of these methods. Amongst the mechanical means used to prevent human-pathogen contact are air filters that per se may prevent human inhalation of particles greater than the size of a virus or bacteria or, in combination with chemicals means, may kill the pathogen upon contact.

The mechanical means to prevent inhalation of virus and bacteria is achieved by reducing the fabric thread spacing density so the light gap between fibers is smaller than any dimension of a desired virus to be blocked. For example, if a virus is at least 10 μm in its smallest dimension, then a fabric with a light gap of less than 10 μm will keep the virus on one side of it and prevent its inhalation.

It is well known to persons skilled in the art that the mineral copper kills viruses that enter in contact with it. A similar fate happens to occur to a bacterium that enters in contact with silver. A fabric that is made of either copper or silver will kill its corresponding pathogen.

Manufacturing fabrics weaved form copper or silver metal filaments/threads for obvious reasons would not be practical for covering human parts or areas where humans may come in contact with.

### BRIEF SUMMARY OF THE INVENTION

The novel invention comprises a chemically treated fabric that has implanted into its structure copper and silver nanoparticles (“NPs”), which upon coming in contact with a pathogen, kills it, thus preventing contact its transmission to humans.

The novel chemically treated fabrics may be used in a plurality of ways, such as, but not limited to facemasks, bed sheets, lab coats, breathing filters, furniture and appliance covers, etc.

The inventive fabrics are treated with sanitizing products based on NPs to confer them pathogen fighting abilities. A solution with silver NPs can confer a treated fabric bactericidal and fungicidal properties. Similarly, a fabric treated with a copper NPs solution can give the treated fabric antiviral characteristics. By combining these NPs treated fabrics into a plurality of products both antibacterial and antiviral properties can be achieved.

The aforementioned combination of fabrics treated with copper NPs, plus other fabrics treated with silver NPs, is novel and unique, and they may be manufactured into a plurality of products having a fighting ability to inactivate or eliminate bacteria, fungi, and viruses.

Although there are fabrics treated with copper NPs in the market and their properties are taken advantage of, as well as there are fabrics treated with silver NPs and are also used for their anti-pathogenic characteristics, there is currently no combination of both fabrics in products of personal protection.

### BRIEF DESCRIPTION OF THE DRAWINGS/PHOTOGRAPHS

FIG. 1 shows a photograph taken via a scanning-electron microscope of untreated and treated fabrics.

FIG. 2 shows an additional photograph taken via a scanning-electron microscope of untreated and treated fabrics.

FIG. 3 shows a photograph taken via scanning-electron microscope of treated and washed fabric.

FIG. 4 shows a facemask made with fabrics treated with the novel method, whereby the inner fabric was treated with silver NPs and outer fabric with copper NPs.

FIG. 5 shows side by side photographs of a sample of treated and untreated fabric.

### DETAILED DESCRIPTION OF THE INVENTION

The composition material of the fabric substrate to be treated with NPs is irrelevant because the anti-pathogenic characteristics of the treated fabric are not directly linked to the composition of the fabric material. In other words, the substrate fabrics could be made of natural fibers, such as cotton, linen, silk, bamboo, wool, or synthetic fibers or a combination of natural and synthetic fibers. The fibers used to weave the fabric substrate may be combined to form plain weave fabrics, knitted fabrics, or any other method to produce woven or non-woven fabrics, to produce a fabric substrate where the sanitizing NPs can be applied via nanotechnology.

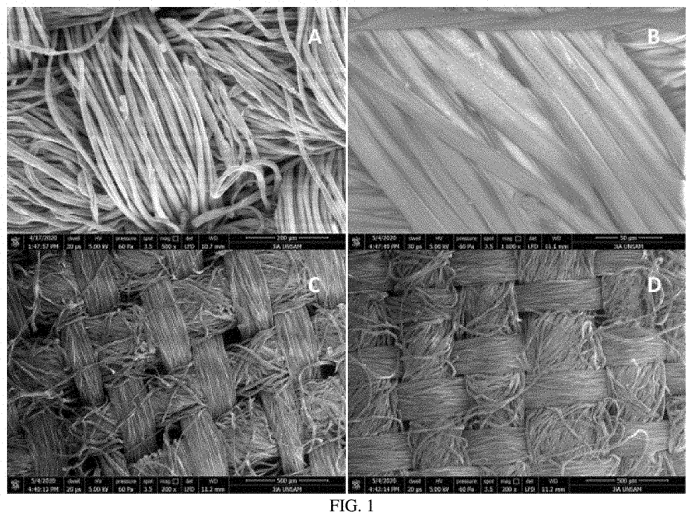

In FIG. 1 the scanning-electron microscope augmented images allow us to appreciate:

A. Untreated fabric

B. Green dyed fabric, treated with polymer and silver NPs

C. Waterproofed fabric, treated with polymer and silver NPs.

D. Waterproofed fabric, dyed blue and treated with polymer and silver NPs

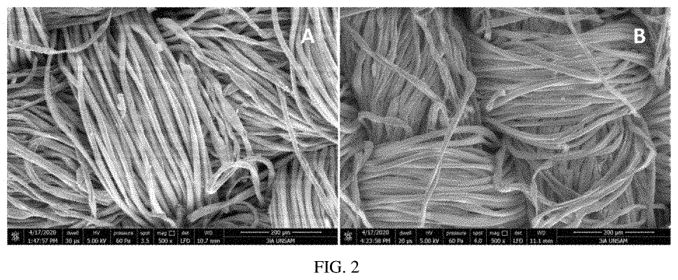

In FIG. 2, the scanning-electron microscope augmented images shows:

A. Untreated fabric

B. Fabric treated with polymer and silver NPs

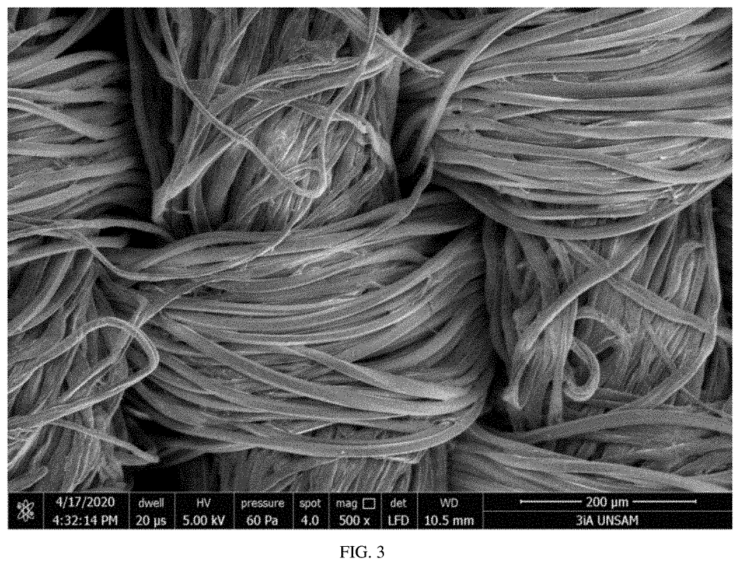

FIG. 3 shows an augment image of a fabric treated with silver NPs after being washed three times.

FIG. 4 shows a drawing of a product, a facemask, made with the treated fabrics.

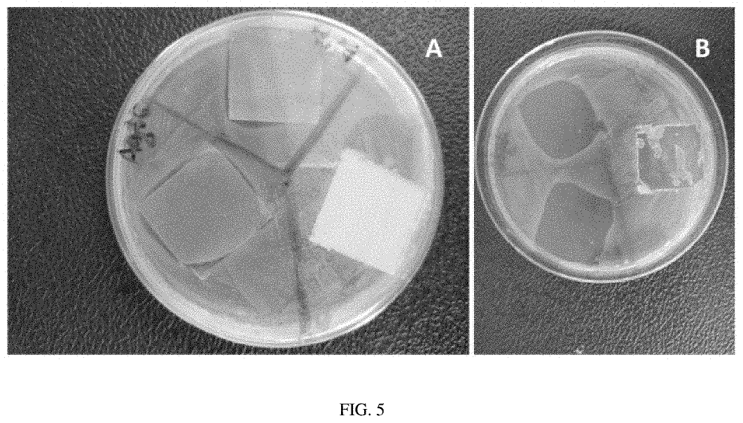

FIG. 5 shows an image of the results of qualitative test (According to JIS L 1902-Halo Method) of antimicrobial activity showing the growth inhibition halo (A) and the growth inhibition under the fabrics (B). Light brown fabrics contain silver NPs. The white fabric is the control fabric without additives. The quantitative test (according to JIS L 1902-Absorption Method) was also carried out.

The invention records the elaboration of the sanitizing products with which the substrate fabrics are treated, the processing of the substrates, the products that are made with them, and especially the combination of fabrics treated with copper NPs and fabrics treated with silver NPs, which is the novelty of the invention.

One of the main advantages of products made with these treated fabrics is that they withstand several washing cycles while maintaining their anti-pathogenic properties. This assertion has been verified in laboratories tests.

The products made with these innovative treated fabrics, such as bed sheets, protective garments, or facemasks, must be washed with water at room temperature, without chlorinated products, by hand and with soap or laundry detergent. Drying must be done without heat and in the shade. The products do not need to be pre-washed for its use, but rather for the personal hygiene of the user.

Since the treated fabrics utilized are washable, a facemask can be used and afterwards washed properly for reuse; this is possible given their germicidal characteristics that prevent the reinsertion of pathogens into the respiratory system causing possible diseases.

### Preferred Embodiment

### Process for Manufacturing a Silver NP Solution

Before starting the processes, the cleanliness of all the containers to be used must be verified and ensured.

A stainless steel reactor, or a reactor with a glazed interior that guarantees its safety, is filled up with demineralized or deionized water of controlled quality (absent of chlorine or other components) at room temperature, normally between 15° C. and 30° C. It is imperative not to mix any other material in the reactor that could contaminate the preparation, such as ferrous metals or others.

The reactor should be equipped with a stirring system suitable for homogeneously mixing the components, a heating system to raise the temperature of its contents, and a cooling system to lower the temperature of its contents.

The steps to obtain a silver NP solution comprises heating the water in the reactor indirectly to guarantee its non-contamination until reaching a temperature range between 65° C. and 95° C. (degrees Celsius).

Turning the reactor's stirring system to its maximum speed and adding 3% to 5% Polyvinyl Alcohol to the heated water. Continuing the vigorous stirring for 5 to 15 minutes so that all the alcohol dissolves homogeneously.

Adding to the mixture between 0.01% and 0.025% of citric acid, maintaining the strong stirring for 5 to 15 minutes.

Cooling the mixture so that the temperature drops within the range between 55° C. and 65° C.

When the temperature reaches the described range, raising the pH with an aqueous sodium hydroxide solution of a concentration no higher than 2.5M to reach a pH with a maximum value of 9.

Reducing the reactor's stirring system to a gentle stirring speed. Dosing very slowly an aqueous solution of silver trioxonitrate with a concentration range of 0.035M to 0.0625M.

At the end of the dosage, adding water at room temperature, maintaining a gentle stirring for 5 to 10 minutes.

The resulting solution must be transferred to innocuous containers of dark color, properly covered, and protected from the light.

### Process for Manufacturing a Copper Np Solution

Before starting the processes, the cleanliness of all the containers to be used must be verified and ensured.

The process comprises the steps of filling up a stainless steel reactor, or a reactor with a glazed interior that guarantees its safety, with demineralized or deionized water of controlled quality (absent of chlorine or other components) at room temperature. It is imperative not to mix any other material in the reactor that could contaminate the preparation, such as ferrous metals or others.

The reactor should be equipped with a stirring system suitable for homogeneously mixing the components, a heating system to raise the temperature of its contents, and a cooling system to lower the temperature of its contents.

Heating the water indirectly to guarantee its non-contamination until reaching a temperature range between 65° C. and 95° C.

Turning the reactor's stirring system to its maximum speed and adding 3% to 7% Polyvinyl Alcohol to the heated water. Continuing the vigorous stirring for 5 to 15 minutes so that all the alcohol dissolves homogeneously.

Adding to the mixture citric acid with a concentration range of 0.2% to 0.6% while maintaining the strong stirring for 5 to 15 minutes.

Diluting copper sulfate pentahydrate in water at 70° C.-90° C. to obtain an aqueous solution of copper sulfate pentahydrate of concentration 0.5M to 1.5M.

Raising the pH with aqueous sodium hydroxide solution of a concentration no higher than 5M to reach a pH between 9 and 11.

Reducing the stirring to a gentle speed, slowly dosing the aqueous solution of copper sulfate pentahydrate into the reactor's mixture.

At the end of the dosage, slowly adding 2% to 3.5% benzalkonium chloride to the mixture, maintaining gentle stirring for 5 to 10 minutes.

The resulting solution must be transferred to innocuous containers of dark color, properly covered, and kept protected from light.

Treatment of Fabrics/Substrates with NPs

A fabric substrate treated with the sanitizing solution must be exposed to a temperature between 150° C. and 200° C. for at least between 1 and 2 minutes.

If using a fabric substrate containing cotton, covalent dyes should be used.

The fabric substrate will be impregnated with the solution by immersion in a pan or vat with the sanitizing product. It must be squeezed, soaked a second time, and squeezed again, to ensure its homogeneity, starting from a dry fabric substrate.

The sanitizing solution applied to the fabric substrate must be polymerized at the temperature and times recommended by the supplier of the applied polymer.

Production of Different Products Based on Fabrics Treated with Copper NPs and Silver NPs

Products made with the two fabric substrates, one treated with copper NPs and another with silver NPs, consist of a sandwich of the two treated fabrics. One or more additional filter layers may be sandwiched between the two treated fabrics to provide for additional air filtration.

The resulting sandwich is cut to shape to manufacture amongst other products, and without limitation, facemasks, patient camisoles, caps, shoe covers, mattress covers, bed sheets, pillow covers, lab coats, furniture and equipment covers, air filters, clothing in general, etc, utilized in hospitals and other aseptic places.

The sum and combination of the protection provided by each fabric substrate treated as above described, provides a protection superior to those existing in the market. This novel invention provides broad spectrum protection against different pathogens that may come from outside or those that the user colonizes with their own breath on the inner side of the facemask.

What it is claimed is:

1. A method to impart antibacterial properties to a substrate fabric, the method comprising the steps of:
a. Providing a clean reactor, the clean reactor having a glazed or a stainless steel interior, a variable speed stirring system suitable for homogeneously mixing components, an indirect heating system to raise the temperature of its contents, and an indirect cooling system to lower the temperature of its contents;
b. Filling the clean reactor with non-chlorinated demineralized or deionized water having a temperature ranging between 15° C. and 30° C.;
c. Heating the water until it reaches a temperature ranging between 65° C. and 95° C.;
d. Vigorously stir the clean reactor's content while adding 3% to 5% polyvinyl alcohol to the clean reactor's heated water;
e. Continuing the vigorous stirring of the clean reactor's content for five to fifteen minutes or until all the polyvinyl alcohol dissolves homogeneously;
f. Adding to the clean reactor 0.01% to 0.025% citric acid while continuing to stir vigorously the clean reactor's contents for and additional five to fifteen minutes;
g. Cooling the clean reactor's contents to a temperature ranging between 55° C. and 65° C.;
h. Rising the clean reactor's content pH with an aqueous sodium hydroxide solution of a concentration no higher than 2.5M to reach a pH with a maximum value of 9;
i. Reducing the clean reactor's stirring to a gentle speed;
j. Slowly dosing an aqueous solution of silver trioxonitrate with a concentration range between 0.035M and 0.0625M to the clean reactor's content;
k. Adding non-chlorinated demineralized or deionized water having a temperature range between 15° C. and 30° C. to the clean reactor's content and maintaining a gentle stirring for an additional five to ten minutes;
l. Heating the clean reactor's content to a temperature ranging between 150° C. and 200° C.;
m. Polymerizing the clean reactor's content at a temperature and time recommended by the supplier of the applied polymer; and
n. Placing a fabric substrate in the clean reactor's content and soaking it for at least two minutes, then squeezing the fabric substrate and soaking it again ensuring its total and homogeneous impregnation with the solution in the clean reactor's content.

2. The fabric substrate of claim 1, wherein the fabric substrate is made from at least one material selected from a group consisting cotton, wool, silk, linen, bamboo, synthetic, and fibers thereof.

3. The fabric substrate containing cotton of claim 2, wherein covalent dyes are used on the fabric substrate.

4. The fabric substrate treated according to claim 1, wherein the treated fabric substrate is used to manufacture a product selected from a group consisting of facemasks, hospital gowns, surgical gowns, lab coats, bed sheets, furniture coverings, air filters, towels, and cleaning cloths.

5. A method to impart antiviral properties to a substrate fabric, the method comprising the steps of:
a. Providing a clean reactor, the clean reactor having a glazed or a stainless steel interior, a variable speed stirring system suitable for homogeneously mixing components, an indirect heating system to raise the temperature of its contents, and an indirect cooling system to lower the temperature of its contents;
b. Filling the clean reactor with non-chlorinated demineralized or deionized water having a temperature ranging between 15° C. and 30° C.;
c. Heating the water until it reaches a temperature ranging between 65° C. and 95° C.;
d. Vigorously stir the clean reactor's content while adding 3% to 7% polyvinyl alcohol to the clean reactor's heated water;
e. Continuing the vigorous stirring of the clean reactor's content for five to fifteen minutes or until all the polyvinyl alcohol dissolves homogeneously;
f. Adding to the clean reactor's content 0.2% to 0.6% citric acid while continuing to stir vigorously for five to fifteen minutes;
g. In a separate clean container diluting copper sulfate pentahydrate in water at 70° C. to 90° C. to obtain an aqueous solution of copper sulfate pentahydrate of concentration 0.5M to 1.5M;
h. Raising the aqueous solution pH with an aqueous sodium hydroxide solution of a concentration no higher than 5M to reach a pH between 9 and 11;
i. Reducing the clean reactor's stirring to a gentle speed, slowly dosing the aqueous solution of copper sulfate pentahydrate into the clean reactor's contents;
j. Slowly adding 2% to 3.5% benzalkonium chloride to the mixture, while maintaining the clean reactor's gentle stirring for five to ten minutes;
k. Heating the clean reactor's content to a temperature range between 150° C. and 200° C.;
l. Polymerizing the reactor's content at a temperature and time recommended by the supplier of the applied polymer; and
m. Placing a fabric substrate in the reactor's content and soaking it for at least two minutes, then squeezing it and soaking it again ensuring its total and homogeneous impregnation.

6. The fabric substrate of claim 5, wherein the fabric is made from at least one material selected from a group consisting cotton, wool, silk, linen, bamboo, synthetic, and fibers thereof.

7. The fabric substrate treated according to claim 5, wherein the treated fabric substrate is used to manufacture a product selected from a group consisting of facemasks, hospital gowns, surgical gowns, lab coats, bed sheets, furniture coverings, air filters, towels, and cleaning cloths.

8. An antibacterial antiviral product, the product comprising a fabric substrate with antibacterial properties processed according to the method of claim 1 sandwiched in conjunction with a fabric substrate with antiviral properties processed according to the method of claim 5.

9. The antibacterial antiviral product of claim 8, wherein the product is one selected from a group consisting of facemasks, hospital gowns, surgical gowns, lab coats, bed sheets, furniture coverings, air filters, towels, and cleaning cloths.

10. The antibacterial antiviral products of claim 9, wherein one or more materials are interspersed with the antibacterial fabric substrate and the antiviral fabric substrate.

11. The antibacterial antiviral products of claim 10, wherein the materials interspersed with the fabric substrates is one material selected from a group consisting of air filters, water-resistant air-permeable filters, impermeable materials.
